(19) United States
(12) Patent Application Publication
(10) Pub. No.: US 2007/0073328 A1
(43) Pub. Date: Mar. 29, 2007

(54) Incrementally expandable balloon

(75) Inventor: Kenneth C. Kennedy II, Clemmons, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc.,, Winston-Salem, NC (US)

(21) Appl. No.: 11/519,279

(22) Filed: Sep. 12, 2006

Related U.S. Application Data
(60) Provisional application No. 60/720,665, filed on Sep. 26, 2005.

(51) Int. Cl. A61M 29/00

(52) U.S. Cl. 606/192

(57) ABSTRACT

A medical device including an expandable member such as a balloon, which includes structure that provides for controlled incremental stepwise radial expansion of the expandable member.

### CROSS-REFERENCE TO RELATED APPLICATIONS

This application claims priority to U.S. Provisional Application Ser. No. 60/720,665, filed Sep. 26, 2005, which is incorporated herein by reference in its entirety.

### FIELD OF THE INVENTION

The present invention relates to medical devices, and specifically relates to expandable medical devices, such as balloons, for use in body lumens.

### BACKGROUND

Various bodily lumens and passages can be afflicted by strictures associated with health problems. For example, in atherosclerosis, deposits in coronary blood vessels create strictures that can impede blood flow and cause increased risk of a heart attack. As another example, various disorders of the gastrointestinal system are associated with strictures in the biliary duct between the gall bladder and the duodenum. Such strictures can cause painful inflammation and, if left untreated, can lead to severe infections and/or cirrhosis.

A variety of devices have been used to treat strictures in different bodily systems. For example, balloon devices have been used. Coronary angioplasty is an example of a procedure utilizing a balloon device. In this procedure, a balloon is expanded within the region of a stricture to obtain or substantially restore a more desirable internal diameter of the blood vessel. In other procedures, a stent is placed in the location of the stricture to maintain the vessel patency. As another example, a physician may dilate a biliary duct stricture to facilitate removal of gall-stones.

Many prior art balloons and other expandable devices are deployed by being inflated/expanded in a gradual, continuous fashion. However, in certain circumstances, it is advantageous to have an expandable device providing a discrete stepwise expansion to one or more predetermined diameters. A number of existing devices have structures and mechanisms for doing so. For example, U.S. Pat. No. 5,304,135 discloses a balloon having a multi-chamber design to confer a staged expansion property to the balloon. As another example, U.S. Pat. No. 5,358,487 discloses a device having annularly arranged balloons wherein inflation of an inner balloon confers a first diameter. The inner balloon ruptures when it is over-inflated, thereby allowing inflation of an outer balloon and conferring a second diameter. The above-described devices are expensive to manufacture and difficult to use. What is needed is an improved balloon device that overcomes the disadvantages of the above-described devices.

### BRIEF SUMMARY

In one aspect, the present invention includes a novel mechanism for staged or incremental expansion that is structurally less complex than those prior art devices.

In another aspect, the present invention includes a medical device having an expandable member, such as a dilation balloon, which is configured to be inflated with an inflation fluid, with a mechanism of the balloon configured to limit radial expansion of the balloon to a first predetermined diameter when the inflation fluid is present at about a first predetermined pressure in the balloon; said mechanism of the balloon providing for a controlled incremental stepwise expansion of the balloon to at least a second predetermined diameter when the inflation fluid is present at about a second predetermined pressure in the balloon. The expandable member may also be configured to expand to a third predetermined diameter. The mechanism may include a mesh in or on a material comprising the surface of the expandable member, or may comprise attachments between surfaces of pleats of the expandable member.

### BRIEF DESCRIPTION OF THE DRAWINGS

FIGS. 1A-1E illustrate side and cross-sectional views of a first embodiment of an incrementally expandable balloon device;

FIG. 1F is a graphic depiction of expansion of the balloon illustrated in FIGS. 1A-1E.

FIGS. 2A-2E show side and cross-sectional views of a second embodiment of an incrementally expandable balloon device;

FIGS. 3A-3H depict side and cross-sectional views of a third embodiment of an incrementally expandable balloon device; and

FIGS. 4A-4D illustrate side views of a semi-rupturable sleeve structure configured for use with an incrementally expandable balloon device.

### DETAILED DESCRIPTION OF THE DRAWINGS AND THE PRESENTLY PREFERRED EMBODIMENTS

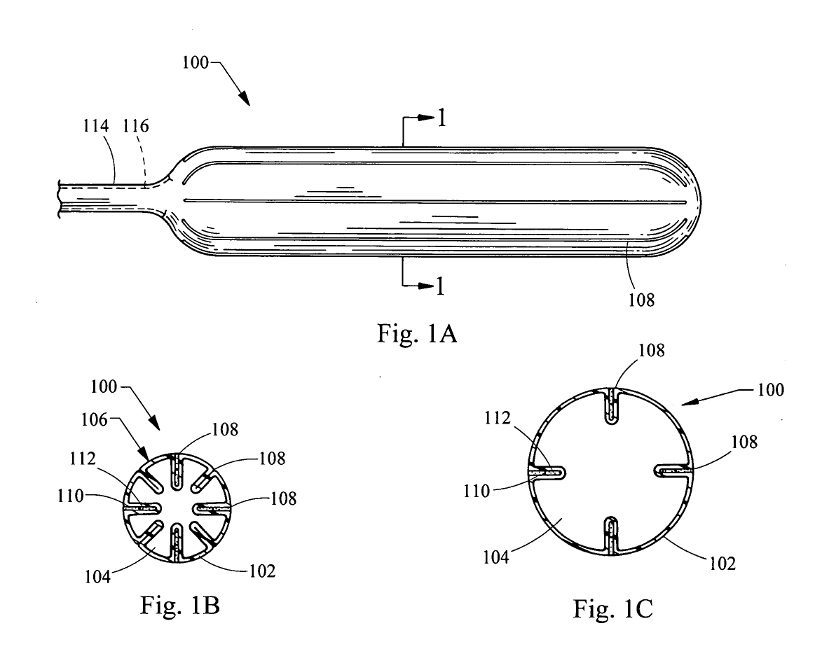

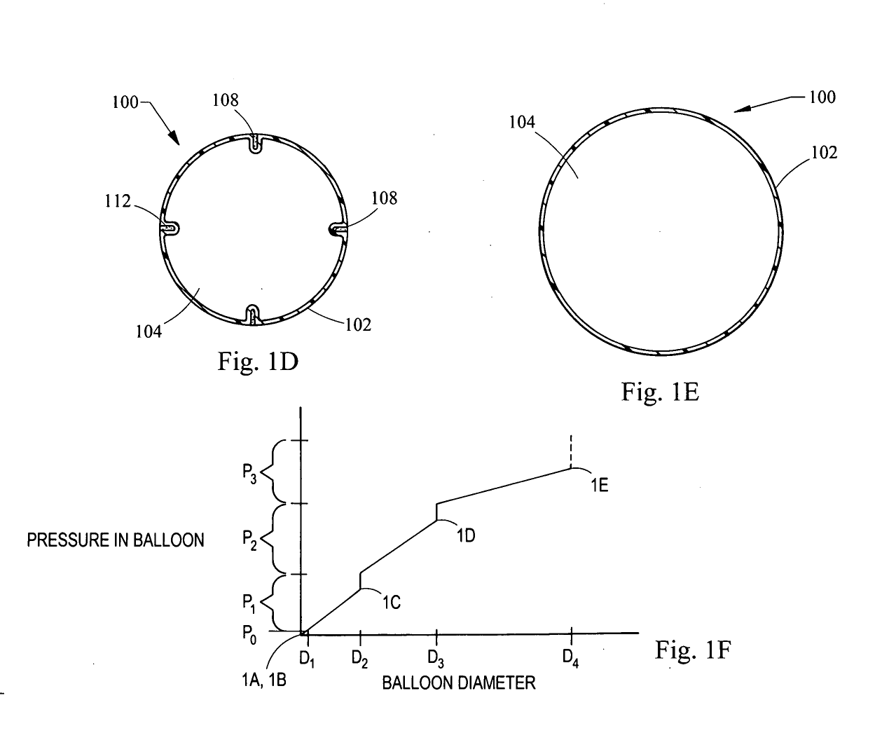

FIGS. 1A-1E illustrate a first embodiment of an incrementally expandable member, embodied as a dilation balloon 100. FIG. 1A is a side view of the balloon 100. The balloon 100 is attached to the distal end of an elongate catheter 114 with an inflation lumen 116 extending therethrough. The proximal end of the catheter 114 is attached to an inflation device (not shown) for supplying an inflation fluid to an interior chamber 104 of the balloon 100. The chamber 104 is configured to be filled with a volume of inflation fluid.

FIGS. 1B-1E are cross-sectional views of the balloon 100 along line 1-1 at different stages of expansion. FIG. 1F shows in graphic form the relationship of the pressure (Pₙ) exerted by the inflation fluid versus the diameter (Dₙ) of the balloon 100. The contours of the graph will vary from that depicted depending upon the elasticity of the balloon components and the incremental expansion mechanism chosen. FIG. 1B illustrates the balloon 100 at an uninflated first diameter (P₀, D₁)(see also FIG. 1F). The balloon 100 includes a wall 102 and the interior chamber 104. The wall 102 comprises a substantially non-compliant/inelastic balloon material and includes a pleated outer surface 106 having a plurality of pleats 108. Some of the pleats 108 have a first adhesive attachment 110 and a second adhesive attachment 112. Each of the first and second adhesive attachments has a predetermined failure strength (and the “adhesive attachments” may alternatively be embodied as bands of material such as suture material or another bridging material with a predetermined failure strength). As will be explained below, the adhesive attachments 110, 112 hold the pleats 108 in a closed configuration such that the diameter of the balloon 100 is restricted during stages of its inflation. Preferably, the adhesive attachments 112 are stronger than the adhesive attachments 110. Those of skill in the art will appreciate that this may be accomplished in several different ways (e.g., use of a stronger adhesive compound, use of a greater quantity of adhesive, different treatment of the balloon surface where the adhesive is applied).

FIG. 1C shows the balloon 100 at a second diameter (D₂) where a first volume of inflation fluid has been directed into the interior balloon chamber 104 sufficient to create a first range of pressure (P₁), radially expanding the balloon 100 to the second diameter (D₂). During the initial expansion, the pleats 108 without adhesive are expanded. The second diameter of the balloon is limited by the first adhesive attachments 110 in the pleats 108. The second diameter (D₂) is the first inflated diameter of the balloon 100 (see also FIG. 1F).

Introduction of a second volume of inflation fluid increases the pressure in the interior chamber 104. When introduction of fluid causes a pressure exceeding the upper end of the first pressure range (P₁), the first adhesive attachments 110 fail and allow the balloon 100 to expand radially to a third diameter (D₃) while inflation fluid is introduced within a second pressure range (P₂). The predetermined failure strength of each of the adhesive attachments may be determined by, for example, selection of a specific adhesive type (independently selected or selected based upon the balloon material), perforation of the adhesive, application of the adhesive in a desired pattern, quantitative control of the amount of adhesive applied, or any other method appropriate for providing a generally predictable failure strength for an adhesive attachment. During the introduction of inflation fluid within the second pressure range (P₂), the third diameter (D₃) of the balloon 100 is limited by the second adhesive attachments 112. FIG. 1D shows the balloon 100 after the adhesive attachments 110 have failed to allow the pleats 108 to partially separate such that the balloon 100 is at the third diameter (D₃), which is limited by the adhesive attachments 112. The third diameter (D₃) is the second inflated diameter of the balloon 100 (see also FIG. 1F).

Introduction of a third volume of inflation fluid further increases the pressure in the interior chamber 104. When introduction of fluid causes a pressure exceeding the upper end of the second pressure range (P₂), the second adhesive attachments 112 fail and allow the balloon 100 to expand radially to a fourth diameter (D₄) while inflation fluid is introduced within a third pressure range (P₃). During the introduction of inflation fluid within the third pressure range (P₃), the diameter of the balloon 100 is limited by the surface compliance of the wall 102. FIG. 1E shows the balloon 100 after the adhesive attachments 110 have failed such that the pleats 108 have fully separated and the balloon 100 is at the fourth diameter (D₄). The fourth diameter (D₄) is the third inflated diameter (see also FIG. 1F).

The above-described structure allows the balloon 100 to expand in a staged, incremental fashion wherein each of the diameters is predetermined and correlates with a predetermined volume and/or pressure of inflation fluid. In alternative embodiments, the balloon 100 may have more or fewer adhesive attachments with a corresponding number of diameters. In other alternative embodiments, the adhesive attachments may include perforated surfaces of material comprising the wall of the balloon. The balloon 100 may be used for dilating a blood vessel, body duct, or some other lumenal structure (e.g., esophagus, pylorus, colon). Alternatively, or in conjunction with dilating a vessel, the balloon may be used to expand a stent for placement in a lumen.

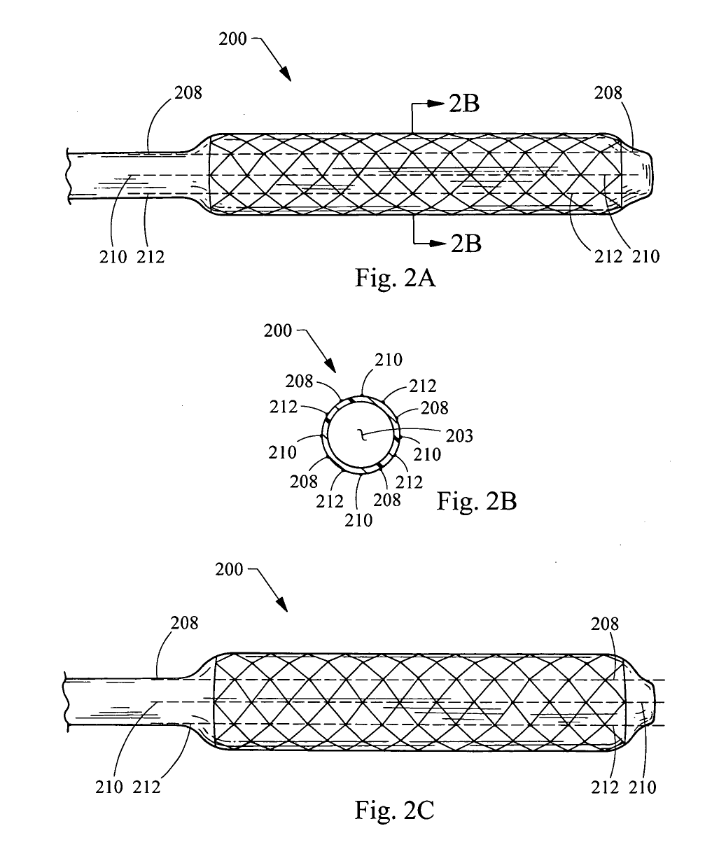

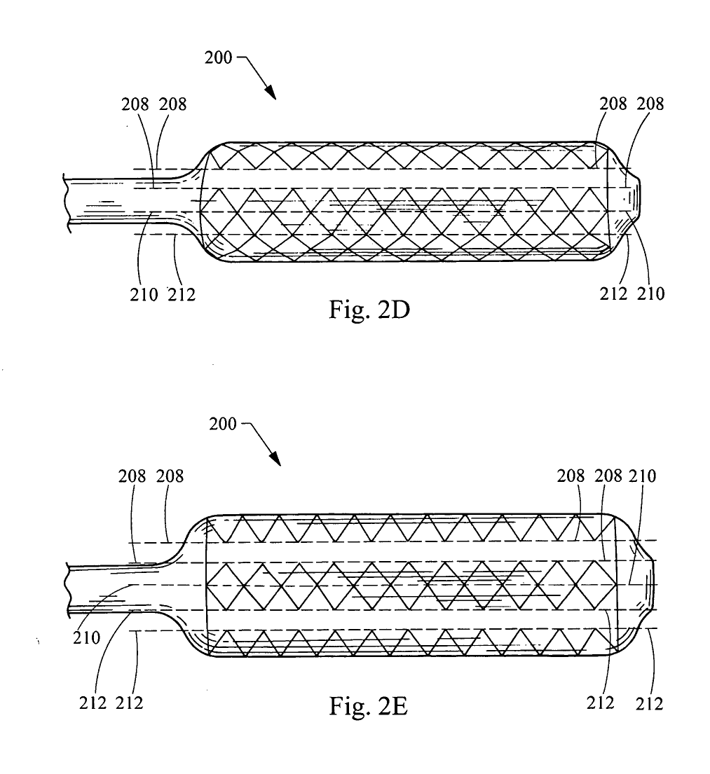

FIGS. 2A-2E illustrate a second embodiment of a balloon 200 of the present invention. FIG. 2A is a side view of the balloon 200, and FIG. 2B is a cross-sectional view along line 2B-2B of FIG. 2A. In FIGS. 2A-2B, the balloon 200 is at an uninflated first diameter. Although FIGS. 2A-2B show the uninflated balloon 200 as having an open interior space, some balloon embodiments, when uninflated will have no open interior space, having been completely evacuated by vacuum or otherwise being formed so as not to provide any interior space when uninflated. The balloon 200 includes a wall 202 comprising a substantially compliant/elastic balloon material and encompassing an inflation chamber 203. A mesh structure 204 is secured along the outer surface 206 of the balloon wall 202. Alternatively, the mesh structure 204 may be incorporated within the construction of the wall 202, configured as a sleeve around the balloon 200 (with ends that remain patent so the sleeve will not come off when portions of the mesh rupture), or at least partially secured to the inner surface of the wall 202. The mesh structure 204 includes a first set of joints 208, a second set of joints 210, and a third set of joints 212 (indicated in FIG. 2A along lines 208-208, 210-210, and 212-212, respectively). Each of the sets of joints 208, 210, 212 preferably has a predetermined failure strength. In the illustrated embodiment, the predetermined failure strength of the second set of joints 210 is indicated as substantially the same as the failure strength of the elastic material comprising the wall 202. The predetermined failure strength of the joints 208, 210, 212 may be established in any suitable manner such as, for example, microperforation, pre-stressing the joint material, providing a thinner/lower diameter in the material at the joints, or forming the joints of a material with a lower tensile strength than the rest of the mesh (wherein the joints 210 have a lower tensile strength than the joints 208). In an alternative embodiment, the joints may have a lower tensile strength and/or a greater malleability such that they stretch (rather than rupture) at a predetermined stress level to allow stepwise expansion.

FIG. 2C shows the balloon 200 at a second diameter where a first volume of inflation fluid has been directed into the inflation chamber 203 sufficient to create a first range of pressure, radially expanding the balloon 200 to the second diameter. The second diameter of the balloon is limited by the intact first, second, and third sets of joints 208, 210, 212.

Introduction of a second volume of inflation fluid increases the pressure in the inflation chamber 203. At a first threshold pressure exceeding the upper limit of the first pressure range, the first set of joints 208 fails and allows the balloon 200 to expand radially to a third diameter while inflation fluid is introduced within a second pressure range. During the introduction of inflation fluid within the second pressure range, the third diameter of the balloon 200 is limited by the intact second and third sets of joints 210, 212. FIG. 2D shows the balloon 200 after the first set of joints has failed to allow the substantially elastic wall 202 to expand radially such that the balloon 200 is at the third diameter. The third diameter is limited by the mesh structure 204, including the intact second and third sets of joints 210, 212.

Introduction of a third volume of inflation fluid further increases the pressure in the inflation chamber 203. At a second threshold pressure exceeding the upper limit of the second pressure range, the third set of joints 212 fails and allows the balloon 200 to expand radially to a fourth diameter while inflation fluid is introduced within a third pressure range. During the introduction of inflation fluid within the third pressure range, the diameter of the balloon 200 is limited by the mesh structure 204, including the second set of joints 210, and by the surface compliance of the wall 202. FIG. 2E shows the balloon 200 after the third set of joints 212 has failed and the substantially elastic wall 202 has been allowed to expand radially so that the balloon 200 is at the fourth diameter. If desired, and if the second set of joints 210 is provided with a rupture strength less than that of the balloon wall 202, the pressure may be increased to a fourth range by introduction of another quantity of inflation fluid, thereby rupturing the second set of joints 210 (not shown). This action would, in stepwise fashion, inflate the balloon 200 to its maximum diameter as limited by its surface compliance.

The above-described mesh structure 204 allows the balloon 200 to expand in a staged, incremental fashion wherein each of the diameters is predetermined. In alternative embodiments, the balloon 200 may have more or fewer joint sets with predetermined failure strengths and with a corresponding number of diameters. The balloon 200 may be used for dilating a blood vessel, body duct, or some other lumenal structure. Alternatively, or in conjunction with dilating a vessel, the balloon may be used to expand a stent for placement in a lumen. In alternative embodiments, more or fewer sets of failing and non-failing joints may be incorporated in the mesh in a manner allowing different incremental stages of expansion/inflation.

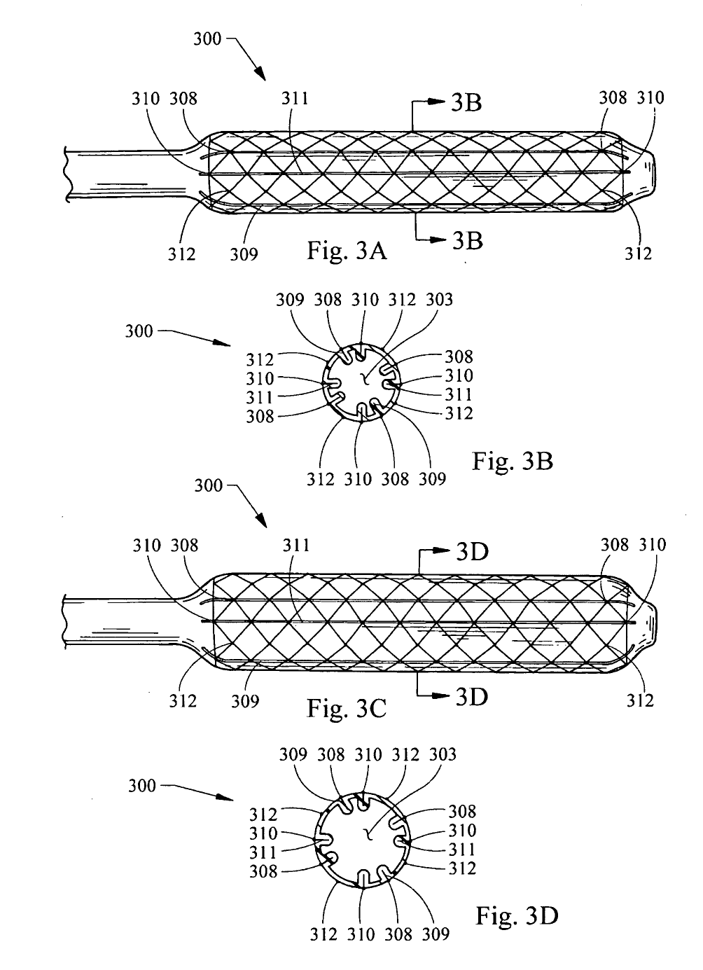

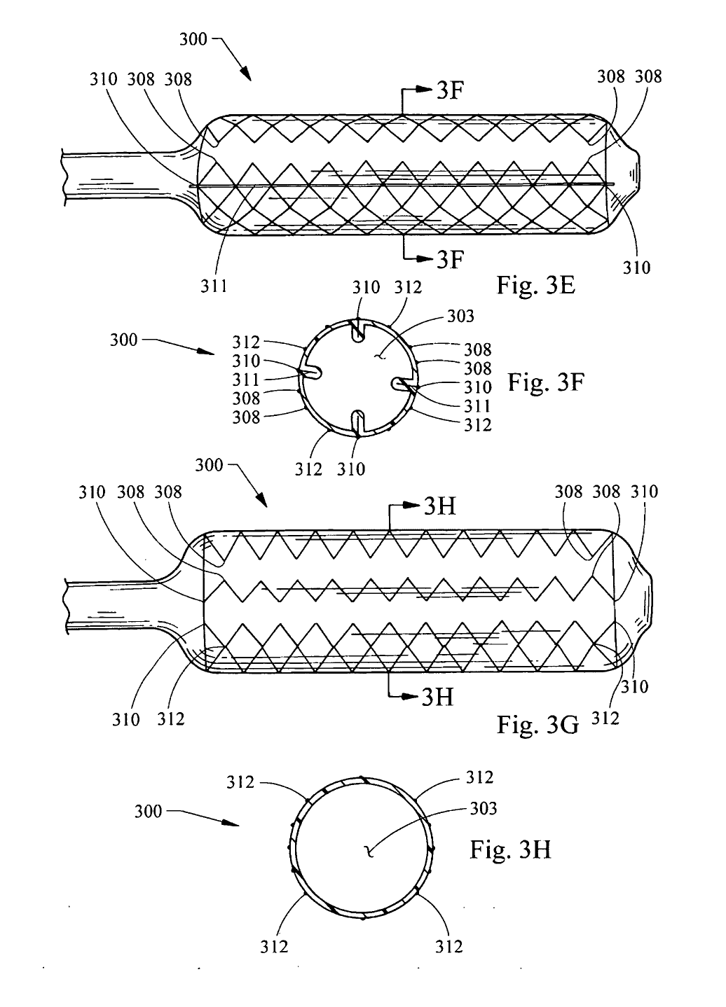

FIGS. 3A-3H illustrate a third embodiment of a balloon 300 of the present invention. FIG. 3A is a side view of the balloon 300, and FIG. 3B is a cross-sectional view along line 3B-3B of FIG. 3A. In FIGS. 3A-3B, the balloon 300 is at an uninflated first diameter. The balloon 300 includes a wall 302 comprising a substantially non-compliant/inelastic balloon material and encompassing an inflation-volume-encompassing chamber 303. A mesh structure 304 is secured to the outer surface 306 of the balloon wall 302. Alternatively, the mesh structure 304 may be incorporated into the construction of the wall 302 or may be secured to an inner surface of the balloon wall 302. The mesh structure 304 includes a first set of joints 308 aligned with a first set of pleats 309, a second set of joints 310 aligned with a second set of pleats 311, and a third set of joints 312. Each of the sets of joints 308, 310, 312 has a predetermined rupture strength. In this embodiment, the predetermined rupture strength of the third set of joints 312 is substantially the same as the rupture strength of the inelastic material comprising the wall 302, although it could be configured to have a lesser rupture strength, thereby allowing for another stepwise stage of expansion, with the final, maximum diameter of expansion being limited by the strength of the wall 302.

FIG. 3C shows a side view of the balloon 300 at a second diameter where a first volume of inflation fluid has been directed into the inflation-volume-encompassing chamber 303 sufficient to create a first range of pressure, radially expanding the balloon 300 to the second diameter. The second diameter of the balloon (which is its first inflated diameter) is limited by the intact first, second, and third sets of joints 308, 310, 312 of the mesh structure 304. FIG. 3D is a cross-sectional view along line 3D-3D of FIG. 3C.

Introduction of a second volume of inflation fluid increases the pressure in the inflation-volume-encompassing chamber 303. At a first threshold pressure exceeding the upper limit of the first pressure range, the first set of joints 308 ruptures and allows the balloon 300 to expand radially to a third diameter while inflation fluid is introduced within a second pressure range. The radial expansion occurs as the pleats 309 are allowed to open upon rupture of the first set of joints 308. During the introduction of inflation fluid within the second pressure range, the third diameter of the balloon 300 is limited by the intact second and third sets of joints 310, 312. FIG. 3E shows a side view of the balloon 300 after the first set of joints has ruptured such that the pleats 309 have opened and the balloon 300 is at the third diameter (which is the second inflated diameter). FIG. 3F is a cross-sectional view along line 3F-3F of FIG. 3E.

Introduction of a third volume of inflation fluid increases the pressure in the inflation volume-encompassing chamber 303. At a second threshold pressure exceeding the upper limit of the second pressure range, the second set of joints 310 ruptures and allows the balloon 300 to expand radially to a fourth diameter while inflation fluid is introduced within a third pressure range. The radial expansion occurs as the pleats 311 are allowed to open upon rupture of the second set of joints 310. During the introduction of inflation fluid within the third pressure range, the diameter of the balloon 300 is limited by the third set of joints 312 and the surface compliance of the wall 302. FIG. 3G shows a side view of the balloon 300 after the second set of joints 310 has ruptured such that the pleats 309 have opened, allowing the balloon 300 to expand radially so that the balloon 300 is at the fourth diameter (which is the third inflated diameter). FIG. 3H is a cross-sectional view along line 3H-3H of FIG. 3G.

Thus, as described, the balloon 300 expands in a staged, incremental fashion wherein each of the diameters is predetermined. In alternative embodiments, the balloon 300 may have more or fewer joint sets with predetermined rupture strengths and with a corresponding number of predetermined diameters. The balloon 300 may be used for dilating a blood vessel, body duct, or some other lumenal structure. Alternatively, or in conjunction with dilating a vessel, the balloon may be used to expand a stent for placement in a lumen.

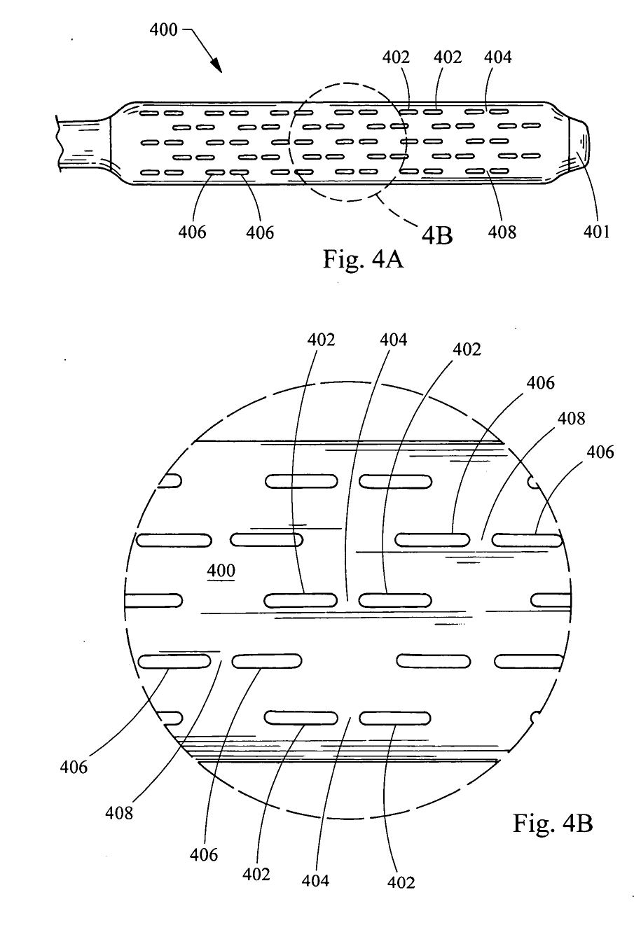

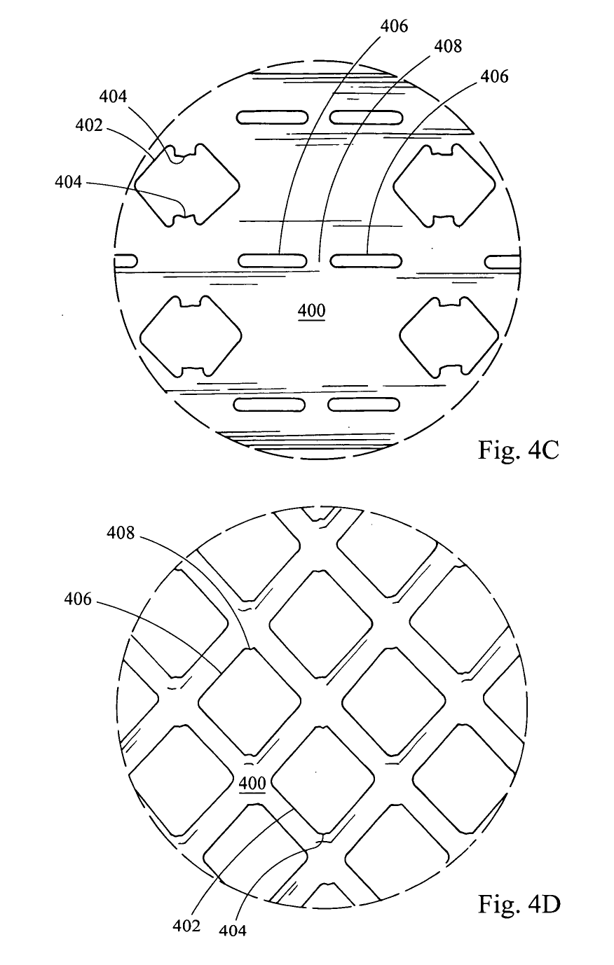

FIGS. 4A-D illustrate an alternative semi-rupturable structure for use in the present invention. As shown in FIG. 4A, a sleeve 400 is provided around a balloon 401. The sleeve 400 includes a first set of slits 402 separated by first rupturable struts 404 and a second set of slits 406 separated by second rupturable struts 408. The first and second sets of slits 402, 406 are positioned in alternating rows. The first struts 404 preferably are weaker than the second struts 408 (e.g., narrower, scored, thinner, decreased material density). FIG. 4B shows a detail view of the sleeve 400, with the struts 404, 408 in an initial, patent state. The struts 404, 408 are configured to rupture in serial fashion when the balloon 401 is expanded. FIG. 4C illustrates the first struts 404 having ruptured (e.g., when the balloon 401 is inflated to a first pressure), allowing the balloon 401 to expand to a first incremental diameter. FIG. 4D illustrates the second struts 408 having ruptured (e.g., when the balloon 401 is inflated to a second pressure), allowing the balloon 401 to expand to a second incremental diameter. As shown in FIGS. 4C-4D, the sleeve 400 maintains patency along its length. Similar to the embodiments described above with reference to FIGS. 2A-3H, a balloon used with the sleeve 400 may be pleated or unpleated, and is enabled by the sleeve 400 to have a controlled, stepwise incremental expansion. The sleeve 400 is preferably formed of a metal or a polymer chosen for its tensile strength relative to a desired balloon expansion force (by increase of internal pressure and diameter) required for rupture of the struts 404, 408.

In alternative embodiments, aspects of the embodiments in FIGS. 2A-4D may be combined with each other and other features. For example, the balloon may be constructed of a semi-elastic material, and have pleats for expansion due to failure to one set of joints having a first predetermined failure strength, while an expansion corresponding to a second set of joints having a second corresponds to an elastic expansion of the balloon. Those of skill in the art will recognize that materials of varying elasticity may be configured for use with balloons of the present invention in a manner consistent with the scope of the present invention.

In other alternative embodiments, the expandable device may be a mechanically deployed basket device constrained by a pleated surface or a mesh structure rather than a balloon that is inflated with an inflation fluid. Each of the expandable devices and the mesh structures of the embodiments described may be constructed from any suitable material. Those of skill in the art will appreciate that, for example, nylon and polyethylene terephthalate (PET) are each suitable for use in forming a balloon or other expandable device as well as in forming a mesh or other structure having portions with a predetermined rupture strength.

It is therefore intended that the foregoing detailed description be regarded as illustrative rather than limiting, and that it be understood that it is the following claims, including all equivalents, that are intended to define the spirit and scope of this invention.

I claim:

1. A medical device comprising:
an expandable balloon configured to be inflated with an inflation fluid; and
a mechanism of the balloon configured to limit radial expansion of the balloon to a first predetermined diameter when the inflation fluid is present at about a first predetermined pressure in the balloon; said mechanism of the balloon providing for a controlled incremental stepwise expansion of the balloon to at least a second predetermined diameter when the inflation fluid is present at about a second predetermined pressure in the balloon.

2. The medical device of claim 1, wherein the mechanism of the balloon comprises a mesh structure.

3. The medical device of claim 2, wherein the mesh structure is substantially intact when the inflation fluid is present at about the first predetermined pressure in the balloon and the mesh structure substantially limits the diameter of the balloon to about the first predetermined diameter; and
wherein at least a portion of the mesh structure is substantially not intact when the inflation fluid is present at about the second predetermined pressure in the balloon; said non-intact state of the at least a portion of the mesh structure allowing the balloon to expand to the second predetermined diameter.

4. The medical device of claim 1, wherein the mechanism of the balloon provides for a controlled incremental stepwise expansion of the balloon to a third predetermined diameter when the inflation fluid is present at about a third predetermined pressure in the balloon.

5. The medical device of claim 4, wherein the mechanism comprises a mesh structure;
wherein the mesh structure is substantially intact when the inflation fluid is present at about the first predetermined pressure in the balloon;
wherein at least one of a first plurality of joining regions in the mesh structure is substantially not intact when the inflation fluid is present at about the second predetermined pressure in the balloon; and
wherein at least one of a second plurality of joining regions in the mesh structure is substantially not intact when the inflation fluid is present at about the third predetermined pressure in the balloon such that the non-intact state of the at least one of a second plurality of joining regions in the mesh structure allows the balloon to expand to the third predetermined diameter.

6. The medical device of claim 1, wherein a surface of the balloon comprises a pleated surface including at least one pleat.

7. The medical device of claim 6, wherein the at least one pleat comprises at least one first attachment between adjacent portions of the pleated surface.

8. The medical device of claim 7, wherein the at least one first attachment is substantially intact when the inflation fluid is present in the balloon at about the first predetermined pressure; and
wherein the at least one first attachment is substantially not intact and the balloon is allowed to expand to the second predetermined diameter when the inflation fluid is present in the balloon at about the second predetermined pressure.

9. The medical device of claim 7, further comprising a second attachment between adjacent portions of the pleated surface.

10. The medical device of claim 9, wherein the at least one first attachment and the second attachment are substantially intact when the inflation fluid is present in the balloon at about the first predetermined pressure;
when the inflation fluid is present in the balloon at about the second predetermined pressure, the at least one first attachment is substantially ruptured; and
when the inflation fluid is present in the balloon at about a third predetermined pressure, the second attachment is substantially ruptured and the balloon is allowed to expand to the second predetermined diameter.

11. A medical device comprising:
an expandable member with an initial diameter, said diameter being configured to be expanded by radially directed force;
a mechanism comprised by the expandable member and configured to limit radial expansion of the expandable member from the initial diameter to one of at least a first expanded diameter and a second expanded diameter; and
wherein the mechanism provides for a controlled stepwise expansion of the expandable member from the initial diameter to the first expanded diameter, and for a controlled stepwise expansion from the first expanded diameter to the second expanded diameter.

12. The medical device of claim 11, wherein the expandable member comprises an inflation balloon and the radially directed force comprises pressure exerted by an inflation fluid within the balloon.

13. The medical device of claim 11, wherein the mechanism comprised by the expandable member comprises a mesh structure.

14. The medical device of claim 13, wherein the mesh structure is substantially intact when an inflation fluid is present at about the first predetermined pressure in the expandable member and the mesh structure substantially limits the diameter of the expandable member to about the first predetermined diameter; and
wherein at least a portion of the mesh structure is substantially not intact when the inflation fluid is present at about the second predetermined pressure in the expandable member; said non-intact state of the at least a portion of the mesh structure allowing the expandable member to expand to the second predetermined diameter.

15. The medical device of claim 11, wherein the mechanism of the expandable member provides for a controlled incremental stepwise expansion of the expandable member to a third predetermined diameter.

16. The medical device of claim 16, wherein the mechanism comprises a mesh structure;
wherein the mesh structure is substantially intact when an inflation fluid is present at about the first predetermined pressure in the expandable member;
wherein at least one of a first plurality of joining regions in the mesh structure is substantially not intact when the inflation fluid is present at about the second predetermined pressure in the expandable member; and
wherein at least one of a second plurality of joining regions in the mesh structure is substantially not intact when the inflation fluid is present at about the third predetermined pressure in the expandable member such that the non-intact state of the at least one of a second plurality of joining regions in the mesh structure allows the expandable member to expand to the third predetermined diameter.

17. The medical device of claim 11, wherein a surface of the expandable member comprises a pleated surface including at least one pleat.

18. The medical device of claim 17, wherein the at least one pleat comprises at least one first attachment between adjacent portions of the pleated surface.

19. The medical device of claim 18, wherein the at least one first attachment is substantially intact when an inflation fluid is present in the expandable member at about the first predetermined pressure; and
wherein the at least one first attachment is substantially not intact and the expandable member is allowed to expand to the second predetermined diameter when the inflation fluid is present in the expandable member at about the second predetermined pressure.

20. The medical device of claim 18, further comprising a second attachment between adjacent portions of the pleated surface.

21. A balloon comprising a capacity to expand in a controlled incremental stepwise fashion to a plurality of predetermined diameters, wherein the balloon is substantially inelastic when it is expanded to one of the plurality of predetermined diameters.
